(19) United States
(12) Patent Application Publication
(10) Pub. No.: US 2008/0095722 A1
(43) Pub. Date: Apr. 24, 2008

(54) Extended Surface Aggregates in the Treatment of Skin Conditions

(75) Inventors: Gregor Cevc, Gauting (DE); Matthias Rother, Grafelfing (DE)

Correspondence Address: WILSON SONSINI GOODRICH & ROSATI, 650 PAGE MILL ROAD, PALO ALTO, CA, 94304-1050, US

(73) Assignee: IDEA AG, Munich (DE)

(21) Appl. No.: 11/667,325

(22) PCT Filed: Nov. 9, 2005
(86) PCT No.: PCT/EP05/11986

§ 371 Date: May 8, 2007

Related U.S. Application Data
(60) Provisional application No. 60/627,546, filed on Nov. 12, 2004.
(60) Provisional application No. 60/642,723, filed on Jan. 10, 2005.
(60) Provisional application No. 60/699,229, filed on Jul. 13, 2005.

(51) Int. Cl. A61K 8/18; A61P 17/00

(52) U.S. Cl. 424/62

(57) ABSTRACT

The invention relates to the use of extended surface aggregates (ESAs) comprising at least one first amphipathic component, which is a basic aggregate-forming component, and at least one second amphipathic component, which decreases aggregate sensitivity to physical stress, including stress created by enforced passage of said ESAs through pores with an average pore diameter at least 50% smaller than the average diameter of the ESAs before said passage, such that the average ESA diameter change induced by such physical stress is reduced by 10% or more, compared to the diameter change induced by such stress in a reference system comprising just the first or just the second aggregate component, in the manufacture of a pharmaceutical preparation for enduring treatment of pathological mammalian skin conditions, including skin irritation, skin inflammation and/or skin damage after topical application, for modifying skin pigmentation and/or for treatment of skin itch.

The invention broadly concerns the application of actives, especially pharmaceutical drug substances, to mammalian, especially human, skin. In one aspect, the invention concerns the treatment of pathological skin conditions including irritation, pain, itching, inflammation and/or skin damage. More specifically the invention concerns the use of extended surface aggregates, including bilayer membranes, based on amphipathic components, especially lipids, in the manufacture of pharmaceutical preparations for the treatment of such pathological skin conditions.

In another aspect, the invention relates to methods and formulations suitable for modifying skin pigmentation in living organisms provided with pigmented skin, and especially in humans and animals. Specifically, the invention is concerned with formulations and methods suitable to induce depigmentation in vivo, without causing skin damage. The invention is also concerned with methods of treating diseases related to hyperpigmentation and pigment cell proliferation.

The skin, including the skin of all mammals, has evolved to become one of the best biological barriers known to mankind. This barrier function is required both to keep necessary substances from leaving the body, and to keep undesired substances from entering the body.

In mammals, this barrier function of the skin is mainly provided by the outermost horny layer of the skin, the stratum corneum.

Many attempts have been made in the past to find transdermal formulations, capable of transporting actives (e.g. pharmaceutical agents) to their destined location in the body (e.g. in muscle tissue or organs) through the intact skin. Generally, such early attempts have been insufficiently effective.

A major breakthrough in transdermal therapy was achieved when it was found that specific mixed lipid bilayers with high permeability and high flexibility characteristics are capable of overcoming narrow, normally confining pores. Often, these take the form of extremely deformable vesicles enclosed by a (generally single) bilayer membrane. The bilayers are formed from amphipathic substances e.g. phosphatidylcholine, which typically form liposomes. Their flexibility is provided by admixture of membrane softening compounds, e.g. surfactants. Vesicles provided with such mixed lipid bilayer membranes can permeate through passages in the skin which would otherwise not even permit the penetration of their constituent molecules. It is assumed that this is based on the opening of initially very narrow (0.4 nm) intercellular hydrophilic channels in the stratum corneum lipid layer by these vesicles, to form hydrophilic pores approx. 20 nm wide, through which the ultradeformable vesicles can then permeate.

This technology is protected by a series of granted patents and patent applications. An early example is EP 0 475 160. A more recent example is WO 2004/032900. A recent scientific article explaining this technology is G. Cevc, A. G. Schätzlein, H. Richardsen and U. Vierl, “Overcoming semi permeable barriers, such as the skin, with ultradeformable mixed lipid vesicles, transfersomes, liposomes or mixed lipid micelles”, Langmuir 2003, 19, 10753-10763. In the literature, vesicles incorporating this technology are often indicated using a trademark owned by the instant applicant, comprising the term “transfersome”. In the context of this description, the term “transfersome” will be used to designate an ultra-deformable vesicle incorporating this technology, as described in the above-mentioned references and commercially available from the applicant. More generally, highly deformable mixed lipid bilayers (whether vesicular or not) will be referred to as “Extended Surface Aggregates” or ESA's.

The published literature describes the use of transfersomes for the transport of actives through the skin, to that part of the body, where their pharmaceutical activity is required. Especially the older transfersome literature stresses the fact that transfersome vesicles penetrate the skin intact, i.e. with the active ingredient carried (as associated with the transfersome material) not only into, but also through and out of the (widened) pores in the stratum corneum, through the underlying epidermal strata and through the dermis, without destruction of the vesicle (although some average size reduction may, in case, be observed). In the treatment of body parts interior of the dermis, this is necessary, to avoid the active being carried off by the blood circulation system, before the destined locus is reached.

### SUMMARY OF THE INVENTION

The present invention is based on the concept of using such mixed lipid bilayer structures or extended surface aggregates, (ESA's) as generally described in the above-mentioned literature (especially in WO 2004/032900) for the treatment of the skin itself, where a skin condition in need of such treatment exists.

Pathological skin conditions do not necessarily involve major structural changes in the skin, and specifically do not generally involve the loss of the stratum corneum's barrier function. Indeed, the pathological skin conditions on which the present invention is mainly focused, leave the barrier function of the stratum corneum basically intact.

Typical such pathological skin conditions include skin irritation, pain, itching, inflammation and/or skin damage, without concurrent loss of the skin's barrier function. Thus, while the skin is not in its natural condition, the skin barrier is functioning. Typical examples include sunburn and other forms of dermatitis.

The skin condition may alternatively have been caused by a treatment that at least partly removes the outer skin cell layers, e.g. erosive laser treatments as used for therapeutic and cosmetic purposes.

The skin condition may be caused by exposure to chemicals, especially skin irritants. The invention e.g. includes the use of ESA's in the therapy of allergies, such as contact allergies.

Generally, reference to therapeutical uses herein is to be understood to include, besides therapy of already existing pathological conditions, also the prevention of such conditions.

In another aspect, the invention concerns the modification of skin pigmentation. It is known that the changes in skin pigmentation can be induced by pharmaceutically active substances.

Skin pigmentation can for example be increased by stimulation of melanocytes, and this may be caused by the application of drugs like cyclophosphamid, MTX, 5-FU, chlofazimin, phenotiazine, thiazide, tetracycline and also NSAIDs (i.e. Non-Steroidal Anti-Inflammatory Drugs).

Depigmentation or hypopigmentation, i.e. the decrease of the concentration of pigments in the skin, can be caused by skin damage (e.g. drug eruptions, contact dermatitis, scarring) induced by various pharmaceutically active substances, including NSAIDs.

In an article by Zailaie, Saudi Med J. 2004 November; 25 (11): 1656-63, in-vitro studies in cell cultures are reported, which appeared to show that in such cell cultures, low concentrations of acetylsalicylic acid stimulate melanocytes, whereas very high concentrations may cause melanocyte apoptosis.

To the Applicant's knowledge, it has not yet been reported that actives such as NSAIDs can induce depigmentation in vivo, in human or animal skin that has not initially been damaged by the drug.

It has now surprisingly been found in the context of a clinical trial, as described below, that transfersome preparations of NSAIDs as described herein can induce profound depigmentation (or hypopigmentation) in vivo, in the absence of any skin damage. Without wishing to be bound to any theory, it is presently assumed that the unparalleled efficacy of transfersomes (and other such amphipathic aggregates, as e.g. described in U.S. Ser. No. 10/357,617) in transporting actives through the stratum corneum, to (and beyond) the deeper strata of the skin, creates exposure of the melanocytes to such high local concentrations of active, that impairment of melanocyte function or even apoptosis can be induced.

Formulations suitable for providing this depigmentation effect include the ones described in above-mentioned U.S. patent application Ser. No. 10/357,617.

Methods of treatment in accordance with this invention include the application of such formulations onto the skin to be treated for extended time periods, up to several days or even weeks, as found necessary.

This invention is useful where treatment of hyperpigmentation or melanocyte dysfunction is desired.

Another potential use of the invention is in the treatment of undesired pigmentation. It is expected that by suitably selecting the pharmaceutically active substance, by selecting its concentration in the formulation and by selecting the time period of treatment, very different effects can be achieved, ranging from a persistent general hypopigmentation, which might just meet cosmetical needs, through the treatment of melasma and melanoma. It is expected that at suitably high active concentrations and suitably long treatment, apoptosis (cell-death) of melanocytes exposed to the treatment can be induced, so that it is possible that undesired growth of melanocytes can be reduced, or noxious melanocyte populations may indeed be entirely removed, which could provide a treatment for e.g. melanoma.

### DEFINITIONS

In the present invention, the general terms employed hereinbefore and hereinafter have the following meanings.

The term “active” means a pharmaceutical active or drug.

The term “aggregate” denotes a group of more than just a few amphipaths of similar or different kind. Typically, an aggregate referred to in this invention contains at least 100 molecules, i.e. has an aggregation number nₐ>100. More often aggregation number is nₐ>1000 and most preferably nₐ>10.000. An aggregate comprising an aqueous core surrounded with at least one lipid (bilayer) membrane is called a lipid vesicle, and often a liposome.

The term aggregate “adaptability” is defined in this document as the ability of a given aggregate to change easily and more or less reversibly its properties, such as shape, elongation ratio, and surface to volume ratio. Adaptability also implies that an aggregate can sustain unidirectional force or stress, such as a hydrostatic pressure, without significant fragmentation, as is defined for the “stable” aggregates. An easy and reversible change in aggregate shape furthermore implies high aggregate deformability and requires large surface-to-volume ratio adaptation. For vesicular aggregates, the latter is associated with material exchange between the outer and inner vesicle volume, i.e. with at least transient vesicle membrane permeabilisation. The experimentally determined capability of given aggregate suspension to pass through narrow pores in a semi-permeable barrier thus offers simple means for functionally testing aggregate adaptability and deformability (vide supra), as is described in the Practical Examples.

To assess aggregate adaptability it is useful to employ the following method:
1) measure fluxjₐ of aggregate suspension through a semi-permeable barrier (e.g. gravimetrically) for different transport-driving trans-barrier pressures delta p;
2) calculate the pressure dependence of barrier penetrability P for given suspension by dividing each measured flux value with the corresponding driving pressure value: P (delta p)=jₐ (deltap)/delta p;
3) monitor the ratio of final and starting vesicle diameter 2rᵥₑₛ(delta p)/2r_{ves,0} (e.g. with the dynamic light scattering), wherein 2rᵥₑₛ(deltap)/is the vesicle diameter after semi-permeable barrier passage driven by delta p and 2r_{ves,0} is the starting vesicle diameter, and if necessary making corrections for the flow-rate effects;
4) align both data sets P (delta p) vs. rᵥₑₛ(delta p)/r_{ves,0}, to determine the co-existence range for high aggregate adaptability and stability; it is also useful, but not absolutely essential, to parameterise experimental penetrability data within the framework of Maxwell-approximation in terms of the necessary pressure value p and of maximum penetrability value Pₘₐₓ, which are defined graphically in the following illustrative schemes.

It is plausible to sum up all the contributions to a moving aggregate energy (deformation energy/ies, thermal energy, the shearing work, etc.) into a single, total energy. The equilibrium population density of aggregate's energetic levels then may be taken to correspond to Maxwell's distribution, All aggregates with a total energy greater than the activation energy, E f E_{A}, are finally concluded to penetrate the barrier. The pore-crossing probability for such aggregates is then given by:

$$P ( e ) = 1 - erf ( \sqrt{\frac{1}{ⅇ}} ) + \sqrt{\frac{4}{π ⅇ} \cdot exp [ - \frac{1}{ⅇ} ]} ,$$

e being dimensionless aggregate energy in units of the activation energy E_{A}.

It is therefore plausible to write barrier penetrability to a given suspension as a function of transport driving pressure (=driving pressure difference) p (=delta p) as:

$$P ( p ) = {p}_{max} \cdot \{ 1 - erf ( \sqrt{\frac{{p}^{*}}{p}} ) + \sqrt{\frac{4 {p}^{*}}{π p}} \cdot exp [ - \frac{{p}^{*}}{p} ] \} \qquad (* )$$

Pₘₐₓ is the maximum possible penetrability of a given barrier. (For the aggregates with zero transport resistance this penetrability is identical to the penetrability of the suspending medium flux.) p* is an adjustable parameter that describes the pressure sensitivity, and thus the transport resistance, of the tested system. (For barriers with a fixed pore radius this sensitivity is a function of aggregate properties solely. For non-interacting particles the sensitivity is dominated by aggregate adaptability, allowing to make the assumption: aₐ proportional to 1/p*.)

The formula (*) is used to calculate aggregate adaptability from suspension flux, or more precisely from the corresponding penetrability (=P(p)=Flux/Pressure=Flux/p data).

This formula is explained, in more detail, in our co-pending U.S. application Ser. No. 10/357,618 “Aggregates with increased deformability, comprising at least three amphipaths, for improved transport through semi-permeable barriers and for the non-invasive drug application in vivo, especially through the skin”, the disclosure of which is incorporated herein by reference.

The term “apparent dissociation constant” refers to the measured dissociation (i.e. ionisation) constant of a drug. This constant for many drugs, including NSAIDs, is different in the bulk and in the homo- or heteroaggregates. For ketoprofen, the pKa in the bulk is approx. 4.4 whereas the pKa value measured above the drug association concentration is approx. 5, and decreases approximately linearly with the inverse ionic strength of the bulk solution. pKa of ketoprofen bound to lipid bilayers increases with total lipid concentration as well, and is approx. 6 and 6.45 in suspensions with 5 w-% and 16 w-% total lipid in a 50 mM monovalent buffer, respectively. For diclofenac, the pKa in the bulk is around 4, whereas for this drug in lipid bilayers pKa ˜6.1 was determined. The bulk pKa reported in the literature for meloxicam, piroxicam, naproxen, indomethacin and ibuprofen is 4.2 (and 1.9), 5.3, 4.2-4.7, 4.5, and 4.3 (or in some reports 5.3), respectively.

The term aggregate “deformability” is closely related to the term “adaptability”. Any major change in aggregate shape that does not result in a significant aggregate fragmentation is indicative of sufficient aggregate deformability, and also implies a large change in the deformed aggregate surface-to-volume ratio. Deformability can therefore be measured in the same kind of experiments as is proposed for determining aggregate adaptability, or else can be assessed by optical measurements that reveal reversible shape changes.

The term “narrow” used in connection with a pore implies that the pore diameter is significantly, typically at least 30%, smaller than the diameter of the entity tested with regard to its ability to cross the pore.

The term “NSAID” (non-steroidal anti-inflammatory drug) typically indicates a chemical entity which acts as cyclooxygenase-1 and/or cyclooxygenase-2 antagonist. Within the framework of this invention lipoxygenase inhibitors are also considered to be part of the class of NSAID's.

Examples include salts of substituted phenylacetic acids or 2-phenylpropionic acids, such as alclofenac, ibufenac, ibuprofen, clindanac, fenclorac, ketoprofen, fenoprofen, indoprofen, fenclofenac, diclofenac, flurbiprofen, pirprofen, naproxen, benoxaprofen, carprofen or cicloprofen; analgesically active heteroarylacetic acids or 2-heteroarylpropionic acids having a 2-indol-3-yl or pyrrol-2-yl radical, for example indomethacin, oxmetacin, intrazol, acemetazin, cinmetacin, zomepirac, tolmetin, colpirac or tiaprofenic acid; analgesically active indenylacetic acids, for example sulindac; analgesically active heteroaryloxyacetic acids, for example benzadac; NSAIDS from the oxicam family include piroxicam, droxicam, meloxicam, tenoxicam; further interesting drugs from NSAID class are, meclofenamate, etc.

The term “phospholipid” means, for example, compounds corresponding to the formula 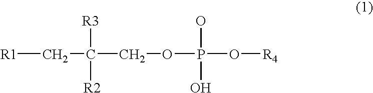
in which one of the radicals R1 and R2 represents hydrogen, hydroxy or C1-C4-alkyl, and the other radical represents a long fatty chain, especially an alkyl, alkenyl, alkoxy, alkenyloxy or acyloxy, each having from 10 to 24 carbon atoms, or both radicals R1 and R2 represent a long fatty chain, especially an alkyl, alkenyl, alkoxy, alkenyloxy or acyloxy each having from 10 to 24 carbon atoms, R3 represents hydrogen or C1-C4-alkyl, and R4 represents hydrogen, optionally substituted C1-C7-alkyl or a carbohydrate radical having from 5 to 12 carbon atoms or, if both radicals R1 and R2 represent hydrogen or hydroxy, R4 represents a steroid radical, or is a salt thereof. The radicals R1, R2, R3, and R4 are typically selected so as to ensure that lipid bilayer membrane is in the fluid lamellar phase during practical application and is a good match to the drug of choice.

In a phospholipid of the formula 1, R1, R2 or R3 having the meaning C1-C4-alkyl is preferably methyl, but may also be ethyl, n-propyl, or n-butyl.

The terms alkyl, alkenyl, alkoxy, alkenyloxy or acyloxy have their usual meaning, expressed in detail in parallel patent application. The long fatty chains attached to a phospholipid can also be substituted in any of usual ways.

A steroid radical R4 is, for example, a sterol radical that is esterified by the phosphatidyl group by way of the hydroxy group located in the 3-position of the steroid nucleus. If R4 represents a steroid radical, R1 and R2 are preferably hydroxy and R3 is hydrogen.

Phospholipids of the formula 1 can be in the form of free acids or in the form of salts. Salts are formed by reaction of the free acid of the formula II with a base, for example a dilute, aqueous solution of alkali metal hydroxide, for example lithium, sodium or potassium hydroxide, magnesium or calcium hydroxide, a dilute aqueous ammonia solution or an aqueous solution of an amine, for example a mono-, di- or tri-lower alkylamine, for example ethyl-, diethyl- or triethyl-amine, 2-hydroxyethyl-tri-C1-C4-alkyl-amine, for example choline, and a basic amino acid, for example lysine or arginine.

A phospholipid of the formula 1 has especially two acyloxy radicals R1 and R2, for example alkanoyloxy or alkenoyloxy, for example lauroyloxy, myristoyloxy, palmitoyloxy, stearoyloxy, arachinoyloxy, oleoyloxy, linoyloxy or linoleoyloxy, and is, for example, natural lecithin (R3=hydrogen, R4=2-trimethylammonium ethyl) or cephalin (R3=hydrogen, R4=2-ammonium ethyl) having different acyloxy radicals R1 and R2, for example egg lecithin or egg cephalin or lecithin or cephalin from soya beans, synthetic lecithin or cephalin having different or identical acyloxy radicals R1 and R2, for Example 1-palmitoyl-2-oleoyl lecithin or cephalin or dipalmitoyl, distearoyl, diarachinoyl, dioIeoyl, dilinoyl or dilinoleoyl lecithin or cephalin, natural phosphatidyl serine (R3=hydrogen, R4=2-amino-2-carboxyethyl) having different acyloxy radicals R1 and R2, for example phosphatidyl serine from bovine brain, synthetic phosphatidylserine having different or identical acyloxy radicals R1 and R2, for example dioleoyl-, dimyristoyl- or dipalmitoyl-phosphatidyl serine, or natural phosphatidic acid (R3 and R4=hydrogen) having different acyloxy radicals R1 and R2.

A phospholipid of the formula 1 is also a phospholipid in which R1 and R2 represent two identical alkoxy radicals, for example n-tetradecyloxy or n-hexadecyloxy (synthetic ditetradecyl or dihexadecyl lecithin or cephalin), R1 represents alkenyl and R2 represents acyloxy, for example myristoyloxy or palmitoyloxy (plasmalogen, R3=hydrogen, R4=2-trimethylammonium ethyl), R1 represents acyloxy and R2 represents hydroxy (natural or synthetic lys6lecithin or lysocephalin, for Example 1-myristoyl- or 1-palmitoyl-lyso-lecithin or -cephalin; natural or synthetic lysophosphatidyl serine, R3=hydrogen, R4=2-amino-2-carboxyethyl, for example lysophosphatidyl serine from bovine brain or 1-myristoyl- or 1-palmitoyl-lysophosphatidyl serine, synthetic lysophosphatidyl glycerine, R3=hydrogen, R4=CH₂OH—CHOH—CH₂—, natural or synthetic lysophosphatidic acid, R3=hydrogen, R4=hydrogen, for example egg lysophosphatidic acid or 1-lauroyl-, 1-myristoyl- or 1-palmitoyl-lysophosphatidic acid).

The term “semipermeable” used in connection with a barrier implies that a suspension can cross transbarrier openings whereas a suspension of non-adaptable aggregates 150-200% larger than the diameter of such openings cannot achieve this. Conventional lipid vesicles (liposomes) made from any common phospholipid in the gel lamellar phase or else from any biological phosphatidylcholine/cholesterol 1/1 mol/mol mixture or else comparably large oil droplets, all having the specified relative diameter, are three examples for such non-adaptable aggregates.

The terms “stable” and “sufficiently stable” mean that the tested aggregate does not change its diameter spontaneously or under relevant mechanical stress (e.g. during passage through a semipermeable barrier) to a practically (most often: pharmaceutically) unacceptable degree. A 2040% change is considered acceptable; the halving of aggregate diameter or a 100% diameter increase is not.

The term “sterol radical” means, for example, the lanosterol, sitosterol, coprostanol, cholestanol, glycocholic acid, ergosterol or stigmasterol radical, is preferably the cholesterol radical, but can also be any other sterol radical known in the art.

The term “surfactant” also has its usual meaning. A long list of relevant surfactants and surfactant related definitions is given in EP 0 475 160 and U.S. Pat. No. 6,165,500 which are herewith explicitly included by reference and in appropriate surfactant or pharmaceutical Handbooks, such as Handbook of Industrial Surfactants or US Pharmacopoeia, Pharm. Eu., etc. Surfactants are typically chosen to be in a fluid chain state or at least to be compatible with the maintenance of fluid-chain state in carrier aggregates.

The term “surfactant like phospholipid” means a phospholipid with solubility, and other relevant properties, similar to those of the corresponding surfactants mentioned in this application, especially in the claims 10 and 1. A non-ionic surfactant like phospholipid therefore should have water solubility, and ideally also water diffusion/exchange rates, etc., similar to those of a relevant non-ionic surfactant.

### DETAILED DESCRIPTION OF THE INVENTION

In the context of this description, the invention will be exemplified in the context of skin analgesia and inflammation, in the context of skin pigmentation, and in treating itch. It is to be understood, however, that the invention is not limited to such treatments, and in fact extends to all preventive and therapeutical treatments of the skin, especially the human skin, which involve correspondingly usable pharmaceutical actives.

In the preferred embodiments, the use of NSAIDs is exemplified. NSAIDs are a preferred class of drugs for practising this invention. It should be understood, however, that other classes of drugs can as well be used in similar treatments of pathological skin conditions. The invention is also not limited to analgesic applications, but extends to the treatment of all kinds of pathological conditions of the mammalian skin.

NSAIDs (“non-steroidal anti-inflammatory drugs”) are a class of drugs with many very well known members. A definition is provided below in the “Definitions” section.

The only currently marketed NSAID formulation in the US for the treatment of any pathological skin condition (Solaraze®) is a diclofenac product for use in actinic ceratosis (praecancerois). This product is reported to cause skin irritation in up to 60% of the treated patients, and seems to be unacceptable for use in inflamed skin conditions.

Sunburn is a model of skin inflammation and a major source of skin pain experienced by humans. It is a clinical response to acute cutaneous solar photo damage after an excessive exposure to ultraviolet, especially UVB light and ranges from mild, painless cutaneous erythema to painful erythemateous skin with associate oedema and blistering. There are no standard treatments for sunburn. A combination of non-pharmacological and pharmacological treatment modalities is currently used to treat sunburn, including topical application of hydrocortisone, but none of these current therapies is considered to be sufficiently efficient.

It is basically known that painful, inflammatory skin conditions such as sunburn and other types of dermatitis, react to the use of NSAIDs, such as indomethacin (Khidbey and Kurban, Journal of Investigative Dermatology 66, 153-156 (1976); Farr and Diffey, British Journal of Dermatology (1986) 115, 453-456; Juhlin and Shroot, Acta derm. Venereol. (Stockh 1992); 72: 222-223). Herein, indomethacin was used in a gel base or in alcoholic solution, and found to provide some inhibition of the appearance of erythema.

Presently, no NSAID formulation is however approved for the treatment of any painful, inflammatory skin condition. In fact, NSAID formulations are contraindicated for the use on irritated and pre-damaged skin. While NSAIDs such as indomethacin may be (limitedly) effective, the irritation potential of corresponding preparations basically prevents use on irritated and predamaged skin.

Besides sunburn there are several comparable painful and often inflammatory skin conditions, which might benefit from anti inflammatory and analgesic treatments. Besides other forms of dermatitis, these include itching, skin damage and skin irritations caused by treatments such as laser therapy.

However (on top of their irritative properties), the known topical formulations are not sufficiently efficient. In the absence of penetration enhancers, such as alcohol, hardly any active actually passes the stratum corneum, which prevents the required pharmaceutical effect. The use of penetration enhancers, especially alcohol, is in itself detrimental where the skin is irritated or damaged, since the use of penetration enhancers then often leads to increased irritation. Even in the presence of penetration enhances, the actives do not penetrate the stratum corneum in sufficient concentrations, to provide the required pharmaceutical efficacy.

Mechanical and electrical methods for providing enhanced transdermal efficiency (iontophoresis, electroporation etc.) are generally unsuitable, because they again increase irritation and pain, where the skin is already irritated and/or damaged.

A need therefore exists for pharmaceutical preparations for the treatment of pathological mammalian skin conditions, which may include skin irritation, skin inflammation and/or skin damage, which makes it possible to transport suitable pharmaceutical actives to their desired locus of activity, and which provides efficient transport of the pharmaceutical active through the stratum corneum, especially without the irritative side-effects of the known preparations.

One object of the invention is therefore to provide pharmaceutical preparations, which may provide a higher efficacy of active penetration through the stratum corneum, for the treatment of pathological mammalian skin conditions, including but not limited to inflammatory conditions, dermatitis, skin irritation, pain, hyperpigmentation and pigment cell proliferation, and itching.

Another important object of the invention is to provide such pharmaceutical preparations which are safe to be used on irritated and/or pre-damaged skin.

Yet another object of the invention is to provide such pharmaceutical preparations which can carry a sufficient drug load through the stratum corneum into the dermis.

In another aspect, the objectives of the invention comprise the provision of new or improved treatments for the above-outlined undesired skin conditions.

In one major aspect of the invention, these objectives are attained by the use of extended surface aggregates (ESAs) comprising at least one first amphipathic component which is a membrane forming lipid component and at least one second amphipathic component which is a membrane destabilising component, whereby the ESA is also capable of penetrating semi-permeable barriers with pores, the greatest diameter of said pores being at least 50% smaller than the average diameter of the ESAs before the penetration, without changing the average ESA diameter by more than 25%, in the manufacture of a pharmaceutical preparation for the treatment of pathological mammalian skin conditions including skin irritation, skin inflammation and/or skin damage.

In a preferred embodiment of the invention, the ESAs comprise at least one third amphipathic component which is also a membrane destabilising component.

In a highly preferred embodiment of the invention, one membrane destabilising component in the extended surface aggregate is itself an active, especially a non-steroidal anti-inflammatory drug (NSAID).

The penetration capability of the ESAs is evaluated using semi-permeable barriers with pores, typically formed by synthetic membranes with known, sufficiently homogenous pore diameters.

The use of such semi-permeable synthetic membranes as a barrier model is described in the art, e.g. in the above mentioned article by Cevc et al. in Langmuir, Volume 19, Number 26, Pages 10753-10763. Such membranes preferably have pore diameters around 20 nm, since this corresponds to the pore size in mammalian skin when the hydrophilic skin pores are widened by the permeation of the inventive extended surface aggregates (ESAs), especially transfersomes.

Generally speaking, ESAs suitable for practicing this invention are known in the art, for different applications. Specifically, such ESAs are described in WO 2004/032900, as above mentioned, the complete contents whereof are therefore hereby incorporated by reference. Some parts of the disclosure of WO 2004/032900 are recited below.

The main difference between this art and the invention lies in the fact that in the reference, the specific use of ESAs to treat pathological mammalian skin conditions is not disclosed, and the preferred parameters which render this use most effective, are not specifically disclosed either. These parameters specifically include the preferred area doses, which differ in the inventive dermatological applications, from the area doses required for transdermal applications in deeper body tissues, such as muscle. The applied area doses suitable for practicing this invention vary, depending on the active used.

One highly preferred active for practicing the present invention is ketoprofen. Ketoprofen is especially preferred, since it is both a Cox 1 and Cox 2 inhibitor and inhibits lipoxygenase activity, so that it can reduce prostaglandin and leucotriene mediated inflammatory reactions.

Typical applied area doses for ketoprofen on human skin are above 0.005 mg per cm² of skin area, more preferably above 0.01 mg and even more preferably lie at 0.02 mg per cm² of skin area or above.

Typically, the applied area dose will not exceed 1 mg per cm², more preferably 0.5 mg per cm² and even more preferred, not more than 0.25 mg per cm².

In presently preferred embodiments, the applied area dose is between 0.01 and 0.07 mg, even more preferred between 0.02 and 0.06 mg ketoprofen per cm² of human skin. 0.06 mg/cm² is a highly preferred applied area dose.

Similar applied area doses may be used for diclofenac, flurbiprofen, piroxicam and other oxicam actives such as meloxicam, tenoxicam etc., as well as other actives with a potency comparable to ketoprofen.

Applied area doses for other NSAIDs, including indomethacin, ketorolac, ibuprofen and naproxen, would be higher, preferably up to and including 10 times higher than the above values given for ketoprofen. For other actives such as salicylates, pyrazalone derivatives (phenylbutazone etc.) or tolmetine, applied area doses would be even higher, up to and including 100 times the above given range for ketoprofen.

The formulations used will generally be as little skin irritating as possible. The ESAs used in accordance with this invention are by definition provided with transdermal activity, which involves the widening of skin pores and therefore some active interference with the epidermis. They generally do not need added penetration enhancers in order to perform. It is therefore possible, and also desirable, to keep the use, and respective concentration, of chemical skin irritants as components of these systems, as low as possible. Thus, formulations using e.g. very little alcohol or no alcohol (especially ethanol) as possible, may be beneficial.

The same applies with respective other potentials skin irritants.

The relatively small applied area does of this invention assist in avoiding skin irritation caused by the pharmaceutical preparation. The preferred use of low dosage formulations such as spray formulations contributes to irritation avoidance.

Quite detailed recommendations on the preparation of inventive combinations are given in EP 0 475 160 and U.S. Pat. No. 6,165,500, which are herewith included by reference, using filtering material with pore diameters between 0.01 μm and 0.1 μm, more preferably with pore diameters between 0.02 μm and 0.3 μm and even more advisable filters with pore diameters between 0.05 μm and 0.15 μm to homogenise final vesicle suspension, when filtration is used for the purpose. Other methods of mechanical homogenisation or for lipid vesicle preparation known in the art are useful as well.

The lipids and certain surfactants mentioned hereinbefore and hereinafter having a chiral carbon atom can be present both in the form of racemic mixtures and in the form of optically pure enantiomers in the pharmaceutical compositions that can be prepared and used according to the invention.

To manufacture a pharmaceutical formulation, it may advisable or necessary to prepare the product in several steps, changing temperature, pH, ion strength, individual component (e.g. membrane destabiliser, formulation stabiliser or microbicide) or total lipid concentration, or suspension viscosity during the process.

A list of relevant and practically useful thickening agents is given e.g. in PCT/EP98/08421, which also suggests numerous interesting microbicides and antioxidants; the corresponding sections of PCT/EP98/08421 are therefore included into the present application by reference. Practical experiments have confirmed that sulphites, such as sodium sulphite, potassium sulphite, bisulphite and metasulphite; and potentially other water soluble antioxidants, which also contain a sulphur or else a phosphorus atom (e.g. in pyrosulphate, pyrophosphate, polyphosphate), erythorbate, tartrate, glutamate, etc. or even L-tryptophan), ideally with a spectrum of activity similar to that of sulphites) offer some anti-oxidative protection to said formulations, final selection being subject to regulatory constraints. Any hydrophilic antioxidant should always be combined with a lipophilic antioxidant, however, such as BHT (butylated hydroxytoluene) or BHA (butylated hydroxyanisole).

### EMBODIMENT EXAMPLES

The invention will now be illustrated in more detail, based on the following examples.

### Example 1

In a first embodiment example, a ketoprofen formulation for the topical treatment of painful skin conditions according to the invention is composed as in Table 1:

TABLE 1

| | | Concentration |
|---|---|---|
| Compound | Function | (mg/g) |
| Ketoprofen, EP | Active agent | 23.82 |
| Soy phosphatidylcholine (SPC) | Carrier agent | 71.46 |
| Ethanol 96%, EP | Solvent | 35.00 |
| Polysorbate 80, EP | Carrier agent | 4.72 |
| Sodium hydroxide, EP | Base | 4.10 |
| Disodium phosphate | Buffering agent | 16.39 |
| dodecahydrate, EP | | |
| Sodium dihydrogen phosphate | Buffering agent | 0.66 |
| dihydrate, EP | | |
| Sodium metabisulphite, EP | Antioxidant | 0.50 |
| Disodium edetate, EP | Chelator | 3.00 |
| Butylhydroxyanisole, EP | Antioxidant | 0.20 |
| Methyl parahydroxybenzoate, EP | Preservative | 2.50 |
| Ethyl parahydroxybenzoate, EP | Preservative | 1.70 |
| Propyl parahydroxybenzoate, EP | Preservative | 0.50 |
| Linalool, FCC | Odor masking agent | 1.00 |
| Benzyl alcohol, EP (optional) | Preservative and | 5.25 |
| | stabiliser | |
| Glycerol 85%, EP | Humectant | 50.00 |
| Water, purified, EP | Solvent | 779.20 |
| Total | | 1000.00 |

### Example 2

It will be noted that the composition of Example 1 comprises relevant amounts of lower aliphatic alcohol (ethanol) which may irritate the skin. A presently more preferred embodiment, comprising no ethanol, is shown in Table 2:

TABLE 2

| | | Concen- |
|---|---|---|
| | | tration |
| Compound | Function | (mg/g) |
| Ketoprofen, EP | Active agent | 4.76 |
| Soy phosphatidylcholine (SPC) | Carrier agent | 14.30 |
| Polysorbate 80, EP | Carrier agent | 0.94 |
| Sodium hydroxide, EP | Base | 0.70 |
| Disodium phosphate | Buffering agent | 8.20 |
| dodecahydrate, EP | | |
| Sodium dihydrogen phosphate | Buffering agent | 0.33 |
| dihydrate, EP | | |
| Sodium metabisulphite, EP | Antioxidant | 0.30 |
| Disodium edetate, EP | Chelator | 1.00 |
| Butylhydroxyanisole, EP | Antioxidant | 0.08 |
| Propyl parahydroxybenzoate, EP | Preservative | 1.00 |
| Butyl parahydroxybenzoate, EP | Preservative | 1.00 |
| (optional) | | |
| Linalool, FCC | Odor masking agent | 0.50 |
| Glycerol 85%, EP | Humectant | 20.00 |
| Water, purified, EP | Solvent | 946.89 |
| Total | | 1000.00 |

### Example 3

Another preferred embodiment, with a small ethanol content, has the following composition:

| Compound | Concentration (mg/g) |
|---|---|
| SPC S100 | 14.30 |
| Ketoprofen | 4.76 |
| Tween 80 | 0.94 |
| Ethanol | 3.00 |
| Glycerol | 20.00 |
| Imidazolidinyl urea | 2.50 |
| BHA | 0.04 |
| Na-Metabisulfite | 0.25 |
| EDTA | 3.00 |
| Linalool | 0.20 |
| Na₂HPO₄ × 12 H₂O | 8.34 |
| NaH₂PO₄ × 2 H₂O | 0.27 |
| NaOH | 1.13 |
| Water, purified, EP | 941.27 |
| Total | 1000.00 |

Total lipid concentration is 2 wt %. Active content (Ketoprofen) is 0.476 wt %. The final product has a pH of 7.9.

### Example 4

A clinical trial was carried out, to study the effect of inventive treatments, on pathological skin conditions including pain and inflammation.

The preparation used was as described in Example 1 above.

The study had a randomised, double-blind, placebo and active controlled format. The primary objective was to compare the effects of a pharmaceutical preparation in accordance with this invention, with placebo, on UVB-skin inflammation. The study involved 25 volunteers.

The study included healthy volunteers of skin type II according to Fitzpatrick, aged 18-45 years. All subjects were non-smokers or infrequent smokers (less than 5 cigarettes per day) and willing not to smoke at least one hour before the procedure started. Exclusion criteria comprised sun tanning four weeks prior to study; pregnancy or lactation; dermal and systemic diseases; mental disorders; any other chronic or acute illness requiring treatment, including dysplastic naevi and praecancerosis. Exclusion criteria further comprised subjects who had used immuno-suppressants (e.g. corticosteroids) within two weeks prior to the study, or had a known sensitivity to NSAIDs, a known photo-allergen/light dermatosis, and substance abusers. The measure of the study was the effect on threshold to heat-induced local pain and erythema following specified UVB irradiation.

Further objectives included the comparison with an equal volume of a commercial product containing hydrocortisone-21-acetate (HC), as well as the testing of lower doses of the inventive preparation, and an evaluation of different application regimes—either immediately after UVB irradiation, or with a delay in treatment.

A comparison was made between skin areas receiving no treatment and no irradiation (control); areas receiving 20 μl of the formulation describes in Example 1 above; areas receiving 20 μl placebo, and areas receiving 20 μl of 0.25 wt % solution of hydrocortisone-21-acetate.

While some skin areas received their treatment directly after UVB irradiation another group received their treatment six hours after UVB irradiation.

In a dose finding part of the study, the amount of formulation according to Example 1 above was varied between 20 μl, 10 μl and 5 μl.

Pain threshold was evaluated in degrees centigrade, erythema and oedema were evaluated on a subjective categorical scale from 0 to 4.

In evaluating the study's primary objective, the effect of 20 μl of a preparation according to Example 1 above was compared to placebo on subjects with UVB-induced sunburn and corresponding induced hyperalgesia to heat.

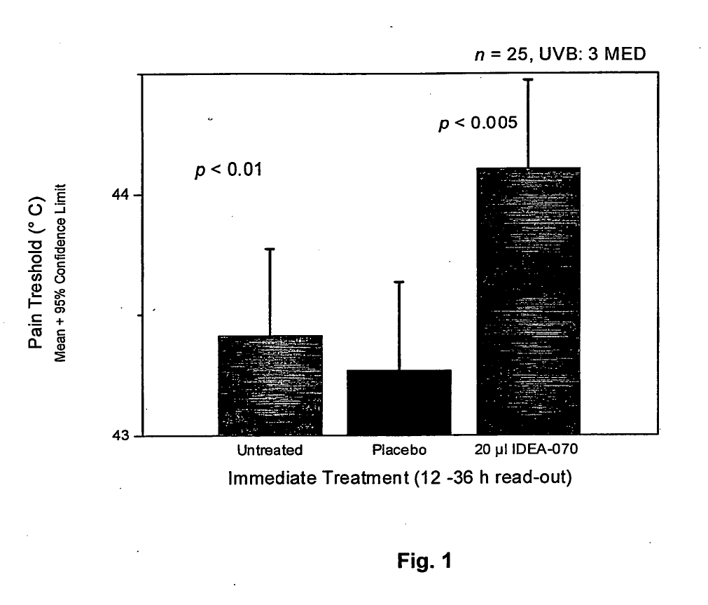

FIG. 1 shows the result for treatment directly after UVB irradiation (3 MED). At 12-36 h read-out, the inventive treatment shows a statistically significant effect over control and placebo.

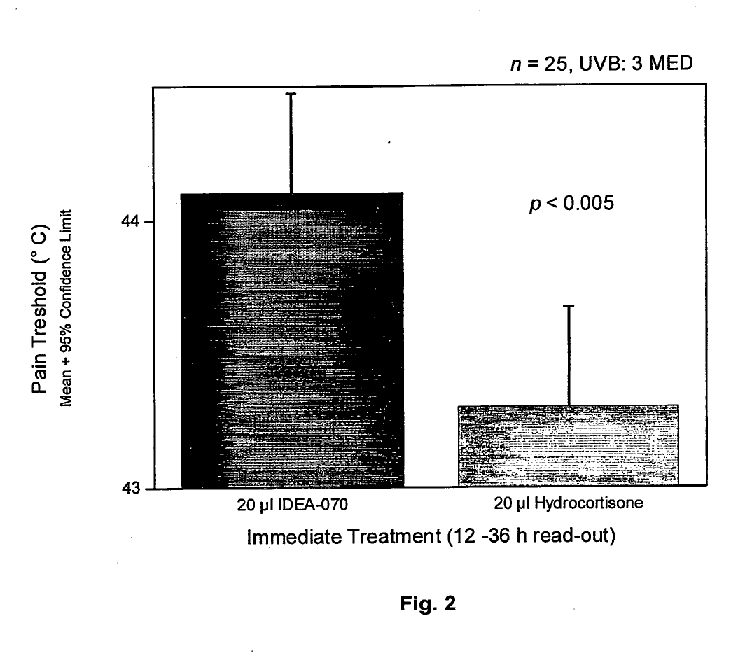

FIG. 2 shows the effect of 20 μl of the Example 1 formulation, on UVB (sunburn) induced hyperalgesia, again for treatment immediately after UVB exposure and at 12-36 h read-out, this time compared to the effect of 20 μl hydrocortisone-21-acetate solution. The effect provided by the invention, as compared to hydrocortisone, is statistically significant superior.

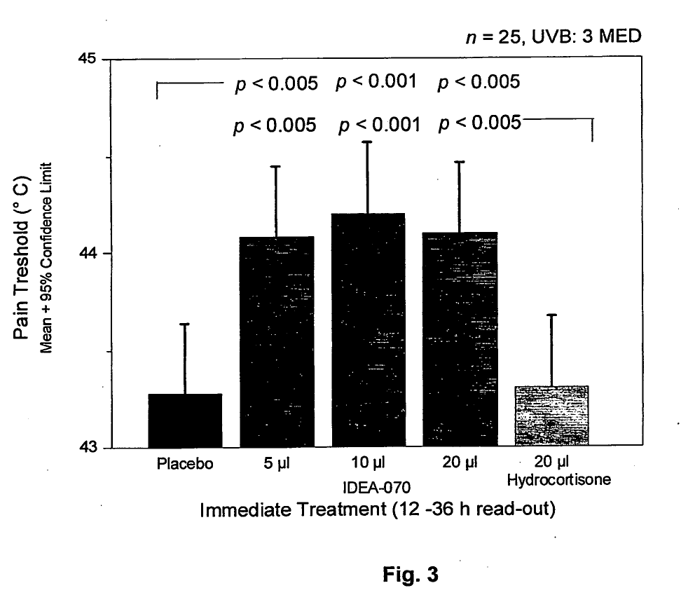

FIGS. 3, 4 and 5 show the results of dose-finding part of the study, again based on the formulation of Example 1, for immediate treatment (3 MED) and read-out at 12-36 h.

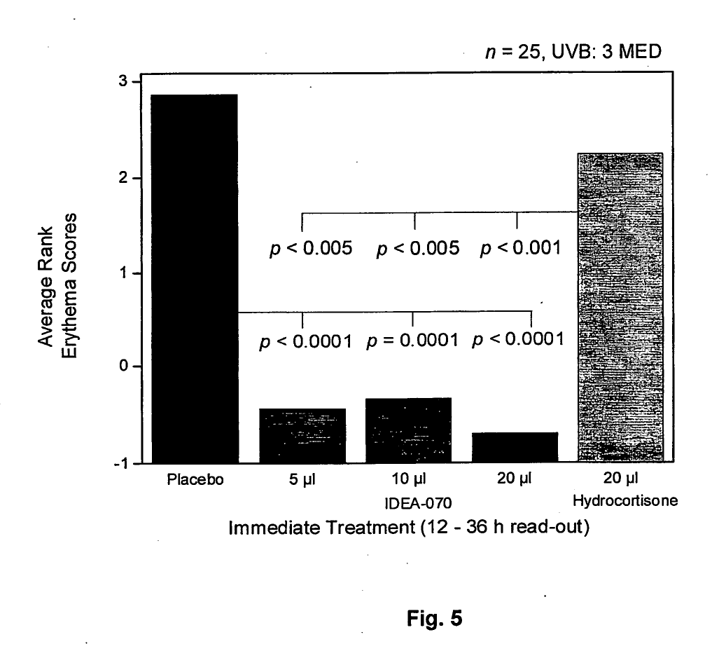

FIG. 5 compares applied doses of 5 μl, 10 μl and 20 μl of the inventive formulation, to, on the one hand, placebo and, on the other hand, 20 μl of 0.25 wt % hydrocortisone solution.

FIG. 3 shows the effect on pain threshold. All three doses tested are significantly superior to placebo and hydrocortisone; there is no relevant effect of dose variation within the tested limits. This may be due to a ceiling effect.

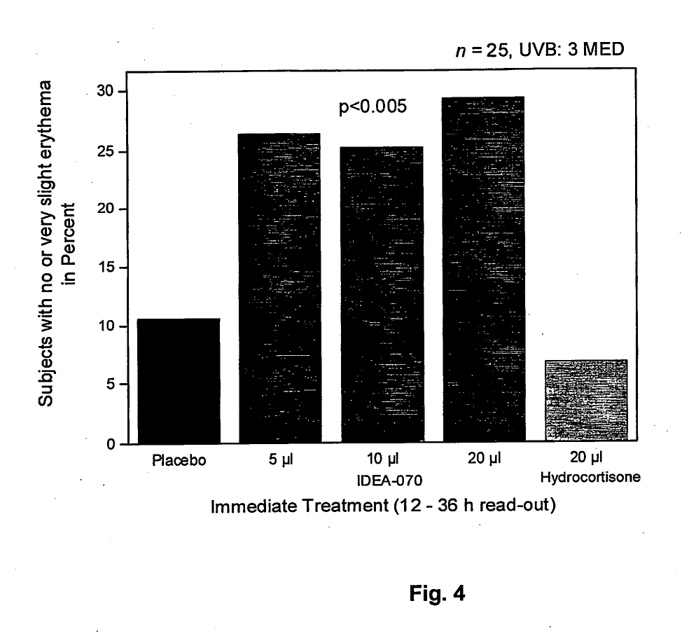

FIG. 4 shows the same comparison, this time in terms of the number of patients where the occurrence of erythema was fully or at least substantially suppressed. Again, the superiority of the invention over hydrocortisone and placebo is statistically significant.

FIG. 5 compares the invention to hydrocortisone and placebo, in terms of the average rank erythema scores, and those patients which produced erythema. It can be seen that only the invention produced any relevant improvement. Again, there is no significant relevance of the dose used.

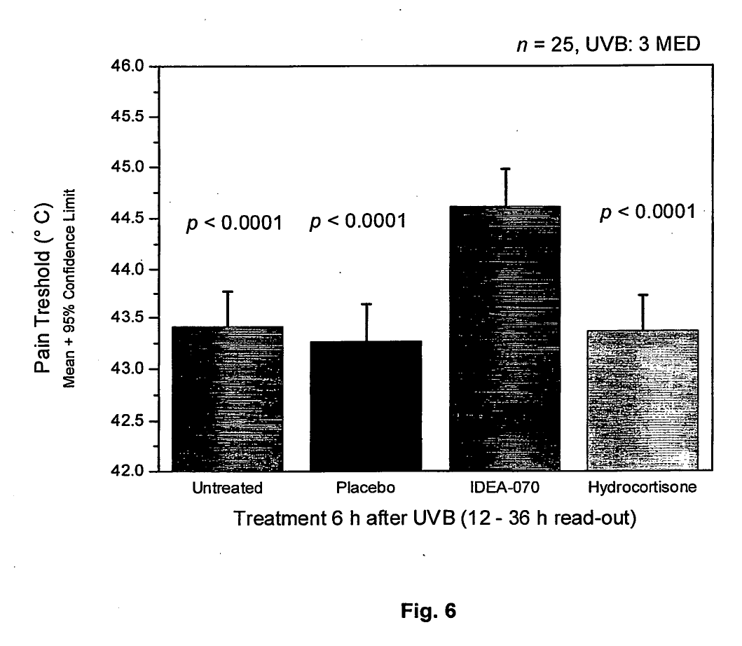

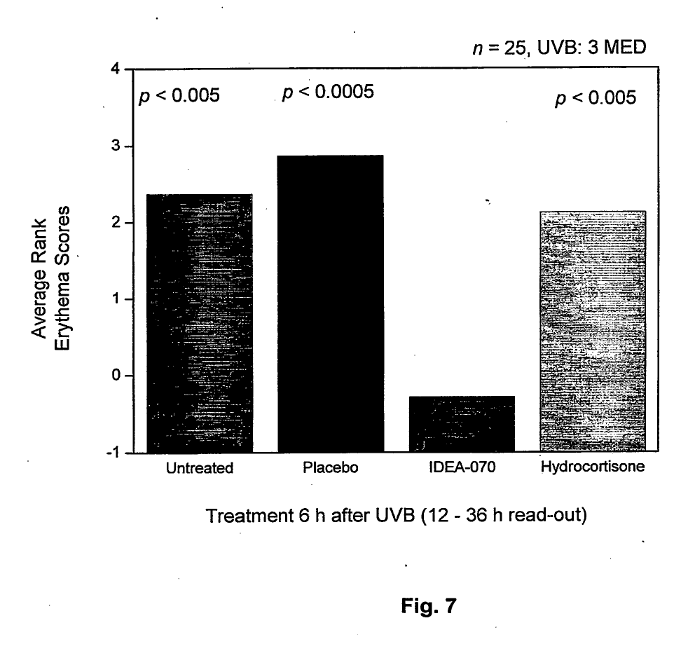

The next aspect evaluated in the study was the effect of the various compared medications, when applied with delay after radiation exposure. All treatments were applied 6 hours after UVB exposure. FIGS. 6 and 7 show the results (read-out at 12-36 h).

Specifically, FIG. 6 showed that after delayed application of 20 μl of the formulation of Example 1, compared to placebo and hydrdcortisone, a statistical significant positive treatment effect on hyperalgesia was experienced by the patients (UVB: 3 MED), whereas hydrocortisone was not significantly different from placebo and control.

In FIG. 7, the same treatments are compared in terms of average rank erythema scores. Again, an effect of any statistical significance is only provided by the invention, whereas hydrocortisone is ineffective at 6 hours delay of treatment.

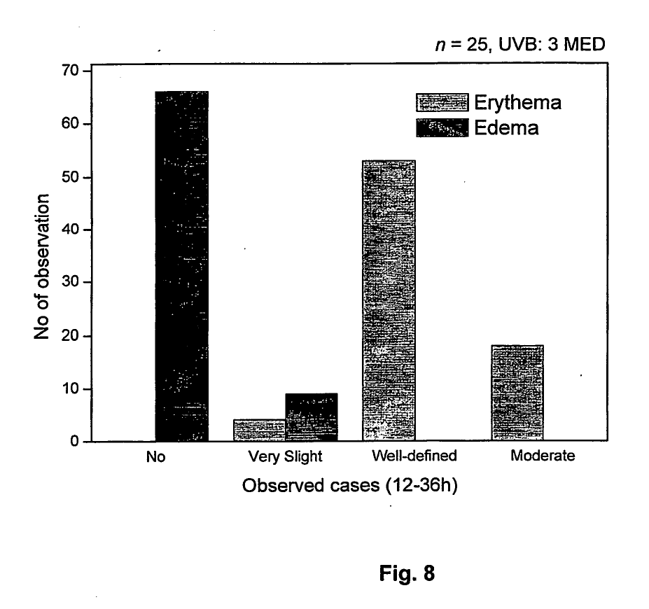

Lastly, FIG. 8 shows the effect of the invention on oedema development. The number of observations of either oedema or erythema after UVB exposure (3 MED) is given, for read-out at 12-36 hours. All subjects developed either no or minor oedema, the majority of subjects developing no oedema at all, when treated with the inventive formulation.

As the study shows, the invention is comparable to the known hydrocortisone treatment in increasing the heat induced pain threshold, where the medication is applied immediately after UVB exposure. This is specifically shown in comparison to untreated but irradiated controls.

In the clinical more relevant situation where the medication occurs with delay (as shown in the 6 hours after UVB exposure tests), only the invention increases the pain threshold, whereas hydrocortisone is ineffective.

The invention prevents erythema development very effectively, both when used directly after UVB exposure and when used with 6 hours delay after the exposure. In both cases, hydrocortisone is ineffective.

The invention effectively prevents oedema formation.

No evidence of dermal intolerance or other adverse events were noted.

### Example 5

Again using basically the formulation of Example 1 above, but at two different concentrations of ketoprofen, a study was carried out on the effect of inventive treatments on contact dermatitis in pigs.

Allergic contact dermatitis was induced in pigs by application of dinitrofluorobenzene on the skin. The resulting contact eczema were evaluated using the criteria in of Table 3:

TABLE 3

| | Criteria | | |
|---|---|---|---|
| | (max. score = 12) | | |
| Score | Extent | Intensity | Induration |
| 0 | no erythema | no erythema | normal finding |
| 1 | barely perceptible | macules of pinhead | nodules of pinhead |
| | eryth. | size | size |
| 2 | slight erythema | lentil-sized macules | doughy lentil-size |
| | | | nodules |
| 3 | moderate erythema | confluent macules | confluent firm |
| | | | nodules |
| 4 | severe erythema | diffuse macules | diffuse hard lesion |

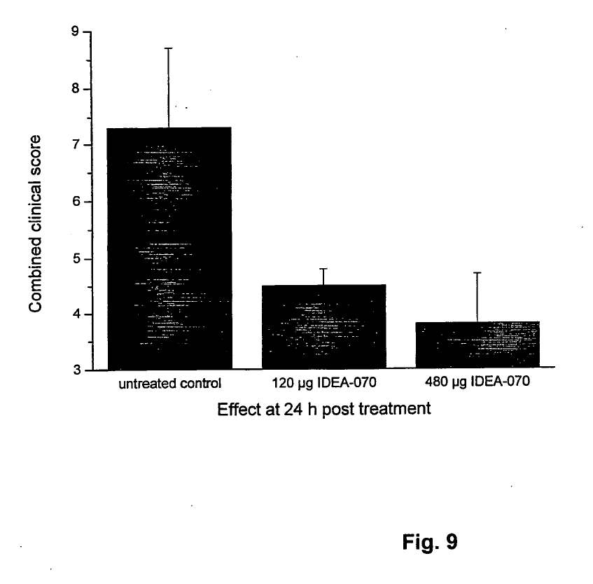

The effects observed at 24 hours post treatment are notable from FIG. 9.

At both applied area doses of 120 μg per cm² and 480 μg per cm², a significant effect was observed, with the higher dose somewhat more effective than the lower one.

### Example 6

In another study, the development of ketoprofen skin concentration (ng/mg) with time was studied at two different applied area doses of a ketoprofen formulation, again as shown in Example 1 above.

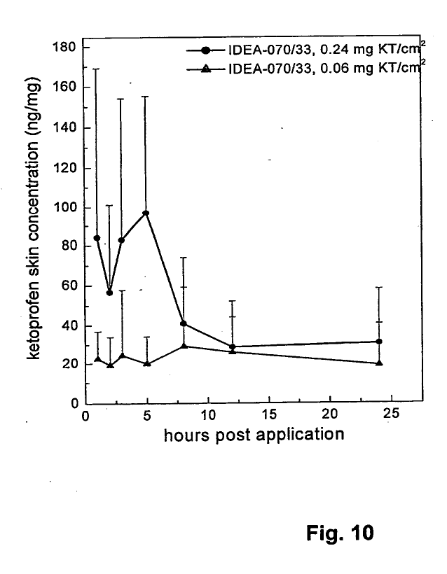

At an applied area dose of 0.24 mg ketoprofen per cm² of pig skin, the skin concentration was significantly higher initially, falling off to basically the same skin concentration as provided by an applied area doses of 0.06 mg per cm² after 8 hours post application. The comparison is shown in FIG. 10.

A comparison with orally administered ketoprofen is shown in Table 4. This lists the applied area dose, the applied total dose and the amount of ketoprofen found in various body tissues after application. The amount in the tissue is given in terms of the AUC (area under the curve) value, for the first 24 hours post application.

The data in Table 4 show the significantly higher skin concentration of active as compared to the concentration in subcutaneous fat, or even deeper lying body tissues such as superficial muscle and deep muscle. As expected, the data indicate that oral ketoprofen provides no topical effect in the skin.

TABLE 4

| | AUC_{1-24 h} [ng × h × mg⁻¹] | | | | | | |
|---|---|---|---|---|---|---|---|
| Product | Ex. 1 | | Ex. 1 | | Ex. 1 | | oral KT |
| Applied area dose (KT/cm²) | 0.5 | mg | 0.24 | mg | 0.06 | mg | n.a. |
| Applied total KT dose | 50 | mg | 24 | mg | 6 | mg | 50 mg |

| | | | | |
|---|---|---|---|---|
| AUC Skin | n.d. | 1022 | 539 | n.d. |
| AUC subcutaneous fat | 710 | 140 | 104 | 11 |
| AUC Superficial muscle | 299 | 89 | 44 | 7 |
| AUC Deep muscle | 267 | 59 | 34 | 9 |

n.d. not determined due to inavailability of tissue samples

### Example 7

Safety of the inventive preparation was studied in a dermal irritation/corrosion study according to Council Directive 92/69/EEC, Annex, Method B.4 in rabbits, which was performed with the clinical trial formulation. The rabbits were treated topically on upper dorsum twice daily ten hours apart for 42 consecutive days with an area: dose of 0.23 mg KT per cm², the same area dose that has also been used in the clinical study Rabbits were Draize-scored (scores from 0 to 4) twice daily prior to test article application for erythema and oedema, also allowing half-value readings.

All animals showed only slight temporary signs of dermal irritation. At the end of the study (day 42) none of the rabbits showed signs of dermal irritation.

Due to the lower drug concentration and overall lower excipient concentrations in formulations as given in Example 2 it is expected that its skin tolerability will be further improved compared to Example 1.

### Example 8

The relatively high drug concentration mediated by the invention's technology might be able to induce therapeutic effects unrelated to the well known prostaglandin-mediated pharmacology. Those effects would be related to direct effects to the nociceptors.

Histamine is often used in the art to induce a neurogenic flare reaction. Recent evidence suggests that there is an itch-specific neural pathway. Human histamine-sensitive C-fibers (small unmyelinated primary afferents) have been characterised by mechanical insensitivity, slow conduction velocity, and huge receptive fields [Schmelz et al., 1997].

The composition of Example 1 was used to study the effectiveness of inventive preparation in reducing histamine-induced itch. This test was part of the study described in Example 4.

The study involved 38 healthy volunteers, who received either an itch-inducing dose of histamine or placebo. Treatment with the formulation of Example 1 showed a trend towards reducing the itching caused by the histamine, as shown by the AUC for Example 1, least square mean: 45.15 (95% cl: 42.46-47.83) compared to placebo, least square mean: 47.83 (95% cl: 45.15-50.52).

### Example 9

The depigmentation effect of the invention was seen in the context of a clinical trial. A 47 year old women with naturally pigmented, brown skin, used a 2.29% ketoprofen gel based on Transfersomes®, as described in U.S. patent application Ser. No. 10/357,617. More specifically, the formulation was closely based on Example 32 of said US patent application, comprising

| Weight-% | |
|---|---|
| 2.290 | Ketoprofen |
| 6.870 | Soy Phosphatidylcholine (SPC) |
| 0.850 | Polysorbate (Tween 80) |
| 3.651 | Ethanol 96% |
| 0.930 | NaOH (sodium hydroxide) |
| 0.235 | Phosphate buffer salts |
| 0.50 | Sodium metabisulphite |
| 0.20 | Butylhydroxytoluene (BHT) |
| 0.100 | Disodium edentate (EDTA) |
| 0.250 | Methyl parahydroxybenzoate |
| 0.525 | Benzyl alcohol |
| 0.100 | Linalool |
| 1.250 | Carbomer (Carbopol 980) |
| 3.00 | Glycerol |
| 79.879 | Water |

The test person was affected by epicondylitis of the right hand, and received concomitant corresponding medication that was unchanged during the time of treatment with the gel. The ketoprofen Transfersome-® gel was repeatedly used over a period of nine days.

Over this time period, a profound depigmentation of the skin topically treated with the ketoprofen gel, became visible. In the skin areas where the gel was applied, the pigmentation was largely destroyed, so that the skin took a white or “bleached” appearance.

After nine days, the use of the transfersome gel was discontinued. The depigmentation effect persisted for more than two months thereafter.

It is assumed that the usefulness of the invention is not limited to ketoprofen, and extends at least to the NSAIDs' class of pharmaceutically active substances. It may be expected that besides ketoprofen, those NSAIDs would be useful in the context of the present invention which show similar depigmentation effectiveness on damaged skin.

It is further expected that beyond NSAIDs, the invention can be used with other drugs that are known to cause depigmentation or hypopigmentation on damaged skin. It is generally assumed that the invention can be practiced with any type of active, in a suitable concentration, that may cause depigmentation, especially by inducing melanocyte apoptosis.

It is also expected that the invention can be used to stimulate pigmentation, where this is desired. This would likely require the application of suitable (low) doses of corresponding actives known to stimulate pigment production by the melanocytes.

The present invention therefore has important potential usefulness in cosmetic as well as medical applications, including the treatment of skin cancer.

Clinical details of the intended treatment will vary, depending on the desired effect, and still need to be studied. Presently, the available evidence is a case report, as described below. Based on general experience and skill, it is however expected that the presently available observations can be extended other patients, and are not limited to any specific patient group.

1. A method for inducing depigmentation, treating hyperpigmentation or treating pigment-cell proliferation, comprising administering to a subject in need or want thereof an effective amount of extended surface aggregates (ESAs) comprising at least one first amphipathic component and at least one second amphipathic component.

2. The method of claim 1, wherein the at least one second amphipathic component is an NSAID.

3. The method of claim 1, wherein the first amphipathic component is a phospholipid.

4. The method of claim 1, wherein the at least one second amphipathic component is a non-ionic surfactant.

5. The method of claim 1, wherein the ESAs further comprise a third amphipathic component, which is chemically different from said second component.

6-7. (canceled)

8. The method of claim 1, wherein the first amphipathic component is a phosphatidylcholine and the second amphipathic component is an NSAID.

9-10. (canceled)

11. The method of claim 1, wherein the first component and the second component differ in solubility by at least 10-fold, on average.

12. The method of claim 5, wherein the second component and the third component differ in solubility, on average, at least 2-fold.

13. The method of claim 1, wherein the total dry mass of the amphipathic components is between 0.01 weight-% and 50 weight-%.

14. The method of claim 1, wherein the ESAs have an average diameter between 15 nm and 5000 nm.

15. The method of claim 1, wherein said ESAs further comprise a lower aliphatic alcohol.

16. The method of claim 2, wherein the ESAs are present in a composition having a bulk pH value that is above the logarithm of the apparent dissociation constant (pKa) of the NSAID in solution and in extended surface aggregates, and the latter pKa is higher than the former.

17. The use method of claim 16, wherein the bulk pH value is between 6.4 and 8.3.

18. The method of claim 1, wherein the ESAs are present in a composition having a bulk ionic strength that is between 0.005 and 0.3.

19. The method of claim 1, wherein the ESAs are present in a composition having a viscosity that is between 50 mPa s and 30,000 mPa s.

20. The method of claim 1, wherein a the at least one first amphipathic component is a phospholipid, the at least one second amphipathic component is an NSAID, and the phospholipid and NSAID are present in a relative molar ratio between 10/1 and 1/2.

21. The method of claim 1, wherein a the at least one first amphipathic component is a phospholipid, the at least one second amphipathic component is a surfactant, and the phospholipid and surfactant are present in a relative molar ratio between 40/1 and 1/4.

22. The method of claim 5, wherein the third amphipathic component is an NSAID and the ESAs are administered at an applied drug dose per unit area of mammalian skin, between 0.0001 mg cm⁻² and 1 mg cm⁻².

23. The method of claim 22, wherein the ESAs are administered at an applied (total) unit dose between 1 mg and 100 mg.

24. The method of claim 22, wherein the ESAs are administered at an applied dose per unit area of mammalian skin between 0.05 mg/cm⁻² and 10 mg/cm⁻².

25. The method of claim 1, wherein the ESAs further comprise a salicylate or a pyrazolene derivative, and the ESAs are administered at an applied drug substance dose per unit area of mammalian skin between 0.5 mg/cm⁻² and 50 mg/cm⁻².

26. (canceled)

27. The method of claim 1, wherein said depigmentation is cosmetic skin depigmentation, or wherein the pigment cell proliferation is undesired.

28. The method of claim 1, wherein the ESAs are administered topically.

29. (canceled)

30. The method according to claim 28, wherein the ESAs are contained in a non-occlusive patch.

31. The method of claim 30, wherein the patch is a component of a kit, in the form of a tube, a spray can or a roller-ball container.

32. (canceled)

33. The method of claim 1, wherein:
the ESAs further comprise an NSAID;
the ESAs are made by loading a suspension of NSAID-free ESAs with an NSAID, the NSAID-free ESAs comprising at least one first amphipathic component and at least one second amphipathic component; and
the ESAs are administered to the subject's skin.

34. The method of claim 2, wherein the NSAID is ketoprofen, ibuprofen, diclofenac, indomethacin, naproxen or piroxicam.

35. The method of claim 3, wherein the phospholipid is a phosphatidylcholine, phosphatidylethanolamine, phosphatidylglycerol, phosphatidylinositol, phosphatidic acid, phosphatidylserines, sphingomyelins, sphingophospholipid, glycosphingolipid, cerebroside, ceramidpolyhexoside, suphatide, sphingoplasmalogene, or ganglioside.

36. The method of claim 4, wherein the non-ionic surfactant is a polyethyleneglycol-sorbitan-long fatty chain ester, a polyethyleneglycol-long fatty chain ester or -ether, a polyhydroxyethylen-long fatty chain ester or -ether, or a surfactant-like non-ionic phospholipid.

37. The method of claim 8, wherein the NSAID is ketoprofen, diclofenac, ibuprofen, indomethacin, naproxen, or piroxicam.

38. The method of claim 5, wherein the first amphipathic component is a phosphatidylcholine and the third amphipathic component is an NSAID.

39. The method of claim 38, wherein the NSAID is ketoprofen, diclofenac, ibuprofen, indomethacin, naproxen, or piroxicam.

40. The method of claim 15, wherein the lower aliphatic alcohol is n-propanol, isopropanol, 2-propanol, n-butanol, 2-butanol, 1,2-propanediol, 1,2-butanediol, or ethanol.

41. The method of claim 17, wherein the bulk pH value is between 6.7 and 8.

42. The method of claim 41, wherein the bulk pH value is between 7 and 7.7.

43. The method of claim 18, wherein the bulk ionic strength is between 0.01 and 0.2.

44. The method of claim 43, wherein the bulk ionic strength is between 0.05 and 0.15.

45. The method of claim 19, wherein the viscosity is between 100 mPa s and 10,000 mPa s.

46. The method of claim 5, wherein the third amphipathic component is an NSAID and the ESAs are present in a composition having a bulk pH value of the preparation that is above the logarithm of the apparent dissociation constant (pKa) of the NSAID g in solution and in extended surface aggregates, and the latter pKa is higher than the former.

47. The method of claim 25, wherein the pyrazolene derivative is phenylbutazone or tolmetine.

48. The method of claim 33, wherein the loading is performed during the day prior to the ESAs' administration.

49. A method for treating skin itching, comprising administering to a subject in need or want thereof an effective amount of extended surface aggregates (ESAs) comprising at least one first amphipathic component and at least one second amphipathic component.

50. The method of claim 49, wherein the at least one second amphipathic component is an NSAID.

51. The method of claim 49, wherein the first amphipathic component is a phospholipid.

52. The method of claim 49, wherein the at least one second amphipathic component is a non-ionic surfactant.

53. The method of claim 49, wherein the ESAs further comprise a third amphipathic component, which is chemically different from said second component.

54. The method of claim 49, wherein the first amphipathic component is a phosphatidylcholine and the second amphipathic component is an NSAID.

55. The method of claim 49, wherein the first component and the second component differ in solubility by at least 10-fold, on average.

56. The method of claim 53, wherein the second component and the third component differ in solubility, on average, at least 2-fold.

57. The method of claim 49, wherein the total dry mass of the amphipathic components is between 0.01 weight-% and 50 weight-%.

58. The method of claim 49, wherein the ESAs have an average diameter between 15 nm and 5000 nm.

59. The method of claim 49, wherein said ESAs further comprise a lower aliphatic alcohol.

60. The method of claim 50, wherein the ESAs are present in a composition having a bulk pH value that is above the logarithm of the apparent dissociation constant (pKa) of the NSAID in solution and in extended surface aggregates, and the latter pKa is higher than the former.

61. The method of claim 60, wherein the bulk pH value is between 6.4 and 8.3.

62. The method of claim 49, wherein the ESAs are present in a composition having a bulk ionic strength that is between 0.005 and 0.3.

63. The method of claim 49, wherein the ESAs are present in a composition having a viscosity that is between 50 mPa s and 30,000 mPa s.

64. The method of claim 49, wherein the at least one first amphipathic component is a phospholipid, the at least one second amphipathic component is an NSAID, and the phospholipid and NSAID are present in a relative molar ratio between 10/1 and 1/2.

65. The method of claim 49, wherein the at least one first amphipathic component is a phospholipid, the at least one second amphipathic component is a surfactant, and the phospholipid and surfactant are present in a relative molar ratio between 40/1 and 1/4.

66. The method of claim 53, wherein the third amphipathic component is an NSAID, and the ESAs are administered at an applied drug dose per unit area of mammalian skin, between 0.0001 mg cm⁻² and 1 mg cm⁻².

67. The method of claim 66, wherein the ESAs are administered at an applied (total) unit dose between 1 mg and 100 mg.

68. The method of claim 66, wherein the ESAs are administered at an applied dose per unit area of mammalian skin between 0.05 mg/cm² and 10 mg/cm⁻².

69. The method of claim 49, wherein the ESAs further comprise a salicylate or a pyrazolene derivative, and the ESAs are administered at an applied drug substance dose per unit area of mammalian skin between 0.5 mg/cm⁻² and 50 mg/cm⁻².

70. The method of claim 49, wherein the ESAs are administered topically.

71. The method according to claim 70, wherein the ESAs are contained in a non-occlusive patch.

72. The method of claim 71, wherein the patch is a component of a kit in the form of a tube, a spray can or a roller-ball container.

73. The method of claim 49, wherein:
the ESAs further comprise an NSAID;
the ESAs are made by loading a suspension of NSAID-free ESAs with an NSAID, the NSAID-free ESAs comprising at least one first amphipathic component and at least one second amphipathic component; and
the ESAs are administered to the subject's skin.

74. The method of claim 50, wherein the NSAID is ketoprofen, ibuprofen, diclofenac, indomethacin, naproxen or piroxicam.

75. The method of claim 51, wherein the phospholipid is a phosphatidylcholine, phosphatidylethanolamine, phosphatidylglycerol, phosphatidylinositol, phosphatidic acid, phosphatidylserines, sphingomyelins, sphingophospholipid, glycosphingolipid, cerebroside, ceramidpolyhexoside, suphatide, sphingoplasmalogene, or ganglioside.

76. The method of claim 52, wherein the non-ionic surfactant is a polyethyleneglycol-sorbitan-long fatty chain ester, a polyethyleneglycol-long fatty chain ester or -ether, a polyhydroxyethylen-long fatty chain ester or -ether, or a surfactant-like non-ionic phospholipid.

77. The method of claim 54, wherein the NSAID is ketoprofen, diclofenac, ibuprofen, indomethacin, naproxen, or piroxicam.

78. The method of claim 53, wherein the first amphipathic component is a phosphatidylcholine and the third amphipathic component is an NSAID.

79. The method of claim 78, wherein the NSAID is ketoprofen, diclofenac, ibuprofen, indomethacin, naproxen, or piroxicam.

80. The method of claim 59, wherein the lower aliphatic alcohol is n-propanol, isopropanol, 2-propanol, n-butanol, 2-butanol, 1,2-propanediol, 1,2-butanediol, or ethanol.

81. The method of claim 61, wherein the bulk pH value is between 6.7 and 8.

82. The method of claim 81, wherein the bulk pH value is between 7 and 7.7.

83. The method of claim 62, wherein the bulk ionic strength is between 0.01 and 0.2.

84. The method of claim 83, wherein the bulk ionic strength is between 0.05 and 0.15.

85. The method of claim 63, wherein the viscosity is between 100 mPa s and 10,000 mPa s.

86. The method of claim 53, wherein the third amphipathic component is an NSAID and the ESAs are present in a composition having a bulk pH value of the preparation that is above the logarithm of the apparent dissociation constant (pKa) of the NSAID in solution and in extended surface aggregates, and the latter pKa is higher than the former.

87. The method of claim 69, wherein the pyrazolene derivative is phenylbutazone or tolmetine.

88. The method of claim 73, wherein the loading is performed during the day prior to the ESAs' administration.

89. A method for treating skin irritation, comprising administering to a subject in need or want thereof an effective amount of extended surface aggregates (ESAs) comprising at least one first amphipathic component and at least one second amphipathic component.

90. The method of claim 89, wherein the at least one second amphipathic component is an NSAID.

91. The method of claim 89, wherein the first amphipathic component is a phospholipid.

92. The method of claim 89, wherein the at least one second amphipathic component is a non-ionic surfactant.

93. The method of claim 89, wherein the ESAs further comprise a third amphipathic component, which is chemically different from said second component.

94. The method of claim 89, wherein the first amphipathic component is a phosphatidylcholine and the second amphipathic component is an NSAID.

95. The method of claim 89, wherein the first component and the second component differ in solubility by at least 10-fold, on average.

96. The method of claim 93, wherein the second component and the third component differ in solubility, on average, at least 2-fold.

97. The method of claim 89, wherein the total dry mass of the amphipathic components is between 0.01 weight-% and 50 weight-%.

98. The method of claim 89, wherein the ESAs have an average diameter between 15 nm and 5000 nm.

99. The method of claim 89, wherein said ESAs further comprise a lower aliphatic alcohol.

100. The method of claim 90, wherein the ESAs are present in a composition having a bulk pH value that is above the logarithm of the apparent dissociation constant (pKa) of the NSAID in solution and in extended surface aggregates, and the latter pKa is higher than the former.

101. The method of claim 100, wherein the bulk pH value is between 6.4 and 8.3.

102. The method of claim 89, wherein the ESAs are present in a composition having a bulk ionic strength that is between 0.005 and 0.3.

103. The method of claim 89, wherein the ESAs are present in a composition having a viscosity that is between 50 mPa s and 30,000 mPa s.

104. The method of claim 89, wherein the at least one first amphipathic component is a phospholipid, the at least one second amphipathic component is an NSAID, and the phospholipid and NSAID are present in a relative molar ratio between 10/1 and 1/2.

105. The method of claim 89, wherein the at least one first amphipathic component is a phospholipid, the at least one second amphipathic component is a surfactant, and the phospholipid and surfactant are present in a relative molar ratio between 40/1 and 1/4.

106. The method of claim 93, wherein the third amphipathic component is an NSAID, and the ESAs are administered at an applied drug dose per unit area of mammalian skin, between 0.0001 mg cm⁻² and 1 mg cm⁻².

107. The method of claim 106, wherein the ESAs are administered at an applied (total) unit dose between 1 mg and 100 mg.

108. The method of claim 106, wherein the ESAs are administered at an applied dose per unit area of mammalian skin between 0.05 mg/cm⁻² and 10 mg/cm².

109. The method of claim 89, wherein the ESAs further comprise a salicylate or a pyrazolene derivative, and the ESAs are administered at an applied drug substance dose per unit area of mammalian skin between 0.5 mg/cm⁻² and 50 mg/cm⁻².

110. The method of claim 89, wherein the ESAs are administered topically.

111. The method according to claim 110, wherein the ESAs are contained in a non-occlusive patch.

112. The method of claim 111, wherein the patch is a component of a kit in the form of a tube, a spray can or a roller-ball container.

113. The method of claim 89, wherein:
the ESAs further comprise an NSAID;
the ESAs are made by loading a suspension of NSAID-free ESAs with an NSAID, the NSAID-free ESAs comprising at least one first amphipathic component and at least one second amphipathic component; and
the ESAs are administered to the subject's skin.

114. The method of claim 90, wherein the NSAID is ketoprofen, ibuprofen, diclofenac, indomethacin, naproxen or piroxicam.

115. The method of claim 91, wherein the phospholipid is a phosphatidylcholine, phosphatidylethanolamine, phosphatidylglycerol, phosphatidylinositol, phosphatidic acid, phosphatidylserines, sphingomyelins, sphingophospholipid, glycosphingolipid, cerebroside, ceramidpolyhexoside, suphatide, sphingoplasmalogene, or ganglioside.

116. The method of claim 92, wherein the non-ionic surfactant is a polyethyleneglycol-sorbitan-long fatty chain ester, a polyethyleneglycol-long fatty chain ester or -ether, a polyhydroxyethylen-long fatty chain ester or -ether, or a surfactant-like non-ionic phospholipid.

117. The method of claim 94, wherein the NSAID is ketoprofen, diclofenac, ibuprofen, indomethacin, naproxen, or piroxicam.

118. The method of claim 93, wherein the first amphipathic component is a phosphatidylcholine and the third amphipathic component is an NSAID.

119. The method of claim 118, wherein the NSAID is ketoprofen, diclofenac, ibuprofen, indomethacin, naproxen, or piroxicam.

120. The method of claim 99, wherein the lower aliphatic alcohol is n-propanol, isopropanol, 2-propanol, n-butanol, 2-butanol, 1,2-propanediol, 1,2-butanediol, or ethanol.

121. The method of claim 101, wherein the bulk pH value is between 6.7 and 8.

122. The method of claim 121, wherein the bulk pH value is between 7 and 7.7.

123. The method of claim 102, wherein the bulk ionic strength is between 0.01 and 0.2.

124. The method of claim 123, wherein the bulk ionic strength is between 0.05 and 0.15.

125. The method of claim 103, wherein the viscosity is between 100 mPa s and 10,000 mPa s.

126. The method of claim 93, wherein the third amphipathic component is an NSAID and the ESAs are present in a composition having a bulk pH value of the preparation that is above the logarithm of the apparent dissociation constant (pKa) of the NSAID in solution and in extended surface aggregates, and the latter pKa is higher than the former.

127. The method of claim 109, wherein the pyrazolene derivative is phenylbutazone or tolmetine.

128. The method of claim 113, wherein the loading is performed during the day prior to the ESAs' administration.
